(19) United States
(12) Patent Application Publication
(10) Pub. No.: US 2006/0275277 A1
(43) Pub. Date: Dec. 7, 2006

(54) Use of coagulation proteins to lyse clots

(76) Inventor: Edward L.G. Pryzdial, Vancouver (CA)

Correspondence Address: David S Resnick;Nixon Peabody, 100 Summer Street, Boston, MA, 02110-2131, US

(21) Appl. No.: 10/551,565

(22) PCT Filed: Apr. 2, 2004
(86) PCT No.: PCT/CA04/00493

§ 371 Date: Feb. 8, 2006

Related U.S. Application Data
(60) Provisional application No. 60/459,647, filed on Apr. 3, 2003.

(51) Int. Cl. A61K 38/54; C12Q 1/37; A61K 31/727

(52) U.S. Cl. 424/94.2; 435/23; 514/56

(57) ABSTRACT

The present invention relates to the use of coagulation proteins for the lysis of blood clots. More specifically, the present invention provides a method for accelerating the dissolution of a blood clot through the administration of at least one coagulation protein comprising a basic C-terminal amino acid, wherein the coagulation protein may be a derivative of Factor X or Factor V or a combination thereof. Pharmaceutical compositions for the treatment and prophylaxis of blood clots are also provided, wherein, the methods and products of the present invention advantageously accelerate clot dissolution while potentially minimizing the adverse side-effects, such as hemorrhaging, seen with other clot dissolving agents. The present invention also provides a method for detecting a fibrinolytic potential in a subject.

### TECHNICAL FIELD

This invention relates to the use of coagulation proteins for the lysis of blood clots.

### BACKGROUND OF THE INVENTION

The flow of blood is regulated by opposing biochemical pathways. A key example is the coagulation pathway, which produces a fibrin clot to seal vascular leaks, and the opposing fibrinolysis pathway, which subsequently-dissolves the clot to ensure normal blood flow is restored. Thrombosis is the disease that results when balance is lost and clotting occurs where it should not. By understanding the molecules involved in maintaining blood flow, drugs have been developed that quickly dissolve these thrombi and reduce the tissue damage caused by oxygen deprivation, especially in acute myocardial infarction. The “clot busters” that have had the greatest impact and are under most intense development are analogues of the natural protein, tissue plasminogen activator (tPA), which is an important initiator of fibrinolysis. However, tPA is not a perfect drug, because it is an active enzyme. Its activity not only helps dissolve the target clot, but systemic rather than strictly localized effects also deplete blood of essential coagulation proteins. This is dangerous because administration of the current thrombolytic drugs often leads to haemorrhage. To avoid some of the complications associated with tPA, novel strategies to better initiate clot lysis are required.

### SUMMARY OF THE INVENTION

In one embodiment of the invention there is provided a method for accelerating blood clot dissolution in a subject in need thereof, the method comprising administering to said subject at least one coagulation protein containing a basic C-terminal amino acid, notably lysine, in an amount effective to enhance dissolving said blood clot.

The administration of a site-specific accelerator of clot lysis rather than tPA, an intrinsically active enzyme, has the advantage of minimizing systemic consequences. This alleviates the hemorrhagic concerns associated with the available thrombolytic drugs.

In an aspect of the invention the coagulation protein is a derivative of Factor X or Factor V or a combination thereof.

In a further aspect of the invention the coagulation protein may be administered to a patient concurrently with a fibrinolytic agent and/or an inhibitor of the coagulation pathway.

In a further embodiment of the invention there is also provided a method for detecting a fibrinolytic potential in a subject, the method comprising: obtaining a blood sample from said subject; and measuring a relative concentration of a coagulation protein comprising a basic C-terminal amino acid or a derivative thereof. Concentration may be measured using a plurality of known protocols as would be understood by one skilled in the art, such as measuring molar concentration, mass concentration, activity, or specific activity.

Accordingly, the present invention provides a method for accelerating blood clot dissolution in a subject in need thereof, the method comprising: administering to said subject at least one coagulation protein comprising a basic C-terminal amino acid in an amount effective to dissolve said blood clot. In a preferred embodiment, the protein is an anionic phospholipid-binding protein. In another preferred embodiment, the subject has a condition selected from: thrombosis, platelet hyperactivity, cardiac ischemia, wound, cardiovascular disease, atherosclerosis, myocardial infarction or a combination thereof. More preferably, the subject is susceptible to said condition and said administration is prophylactic.

In an embodiment, said at least one coagulation protein is a derivative of Factor X. More preferably, said derivative is selected from Factor Xaα, Xaβ, Xaγ, or a combination thereof. In another preferred embodiment, said at least one coagulation protein is a derivative of Factor V. More preferably, said derivative is Factor Va.

In another embodiment of the present invention, said at least one coagulation protein comprises a derivative of Factor X and a derivative of factor V.

According to methods of the present invention, administration comprises administering to the subject a pharmaceutical composition comprising said derivative of Factor X and an acceptable carrier. More preferably, said derivative of Factor X is selected from Xaα, Xaβ and Xaγ or a combination thereof.

In another preferred embodiment of the methods of the present invention, administering comprises administering to the subject a pharmaceutical composition comprising said derivative of Factor V and an acceptable carrier. More preferably, said derivative of Factor V is selected from Va.

In accordance with the methods of the present invention, said pharmaceutical composition further comprises a fibrinolytic agent selected from tissue plasminogen activator, urokinase, streptokinase or a combination thereof. In addition, said pharmaceutical composition may further comprise an inhibitor of thrombin. In a preferred embodiment, said inhibitor of thrombin is selected from hirudin; bivalirudin, lepirudin and heparin or a combination thereof.

In a preferred method of the present invention, said pharmaceutical composition is administered intravenously, intramuscularly, subcutaneously, intraperitoneously or intraarterially or a combination thereof.

The present invention also provides a method for detecting a fibrinolytic potential in a subject the method comprising: (a) obtaining a blood sample from said subject; and (b) measuring a relative concentration of a coagulation protein selected from a coagulation protein comprising a basic C-terminal amino acid, a derivative of a coagulation protein comprising a basic C-terminal amino acid or a combination thereof. In a preferred embodiment, said coagulation protein is selected from a derivative of Factor X or Factor V.

The present invention additionally provides a pharmaceutical composition comprising a coagulation protein for the treatment or prophylaxis of blood clotting, wherein said coagulation protein comprises a basic C-terminal amino acid. More preferably, said coagulation protein is a derivative of Factor X or Factor V or a combination thereof. In a preferred embodiment, said Factor X is selected from Xaα, Xaβ and Xaγ or a combination thereof, and Factor V is selected from Va.

A pharmaceutical composition according to the present invention may additionally comprise a pharmaceutically acceptable carrier, and/or one or more fibrinolytic agents, and/or one or more inhibitors of the coagulation pathway.

### BRIEF DESCRIPTION OF THE DRAWINGS

Further features and advantages of the present invention will become apparent from the following detailed description, taken in combination with the appended drawings, in which:

FIG. 1 is a schematic representation of some of the derivatives of Factor X;

FIG. 2A is plot of clot amount as measured by relative absorbance at 405 nm as a function of time;

FIG. 2B is a plot of the % lysis of clot as a function of Factor-Xay concentration.

FIG. 3 shows electrophoresis gels of fragmentation patterns of Factor X.

### DETAILED DESCRIPTION OF THE PREFERRED EMBODIMENT

It has been recognized that the enzyme directly responsible for dissolving fibrin, plasmin (Pn), can change the function of at least two coagulation proteins, factor Xa (Xa) and factor Va (Va). By limited proteolysis these are converted into accelerators of tPA [Pryzdial, E. L. G., Lavigne, N., Dupuis, N., Kessler, G. E. (1999) Journal of Biological Chemistry 274:8500-8505; Pryzdial, E. L. G. and Kessler, G. E. (1996) Journal of Biological Chemistry 271:16614-16620; and Pryzdial, E. L. G., Bajzar, L. and Nesheim, M. E. (1995) Journal of Biological Chemistry, 270:17871-17877]. This function is only acquired when the Pn-treated Xa and Va are bound to negatively charged phospholipids which are normally localized to the vicinity of a clot. However, the clot itself is the accepted physiological tPA accelerator. Enhanced Pn generation and solubilization of a fibrin clot are thus considered distinct biochemical and physiological processes.

In one embodiment of the present invention there is provided coagulation proteins comprising a basic C-terminal amino acid that significantly accelerates solubilization of blood clots. These coagulation proteins may comprise derivatives of factor X and factor V.

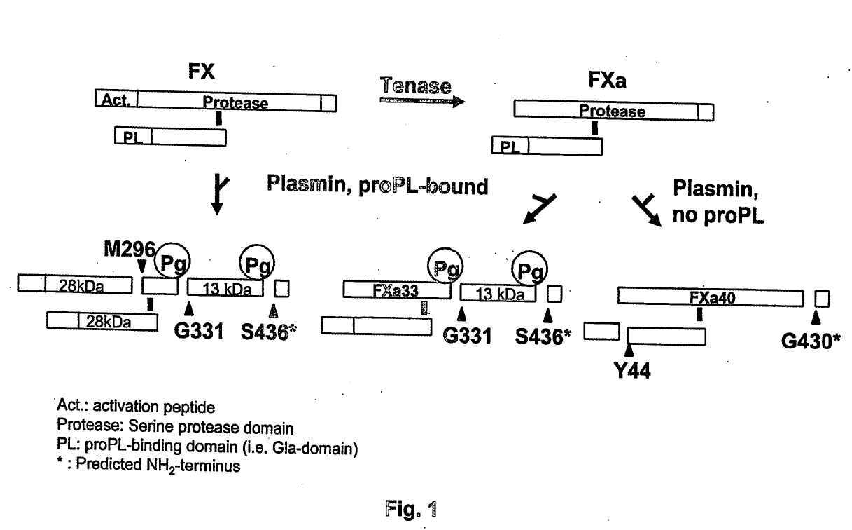

Factor Xa: Several compositions of Factor Xa produced by the proteolytid activity of Pn under different conditions were evaluated for enhancement of clot lysis. Factor Xa and factor X fragments generated by Pn, which we determined earlier (Pryzdial, E. L. G., Lavigne, N., Dupuis, N., Kessler, G. E. (1999) Journal of Biological Chemistry 274:8500-8505 and Pryzdial, E. L. G. and Kessler, G. E. (1996) Journal of Biological Chemistry 271:16614-16620] are summarized in FIG. 1. Binding to procoagulant phospholipid (proPL) alters the cleavage pattern as indicated. When bound to proPL, purified Xa is cleaved twice by Pn to produce fragments of 33, 13 and 3 KDa, which we collectively refer to as Xay. An additional minor product of 28 KDa after prolonged Pn-treatment of Xa (28 KDa) has been observed (not shown) and is likely due to cleavage of Xa33 at Met296, as in X. To determine the non-covalent interactions that form between the Xay fragments, we have used either proPL coated on microtitre plates or large high density multilamellar vesicles (300 μm) as affinity matrices and by electrophoresis found that Xa33 and 13 KDa remain associated to proPL (not shown)(Grundy, J., Hirama, T., MacKenzie, R. and Pryzdial, E. L. G. (2001) Biochemistry 40:6293-6302). Since the entire proPL binding site of Xa is contained within Xa33, this observation demonstrates that Xa33 and Xa13 form a noncovalent heterodimer. Both. Xa33 and Xa13 are predicted by N-terminal sequencing of the successive fragment to contain a C-terminal Lys (K330 and K435, respectively). When Xa is subject to proteolysis by plasmin under conditions that do not facilitate binding to proPL, different peptide bonds are modified as indicated in the diagram.

Compositions of Factor Xa Accelerate Clot Lysis:

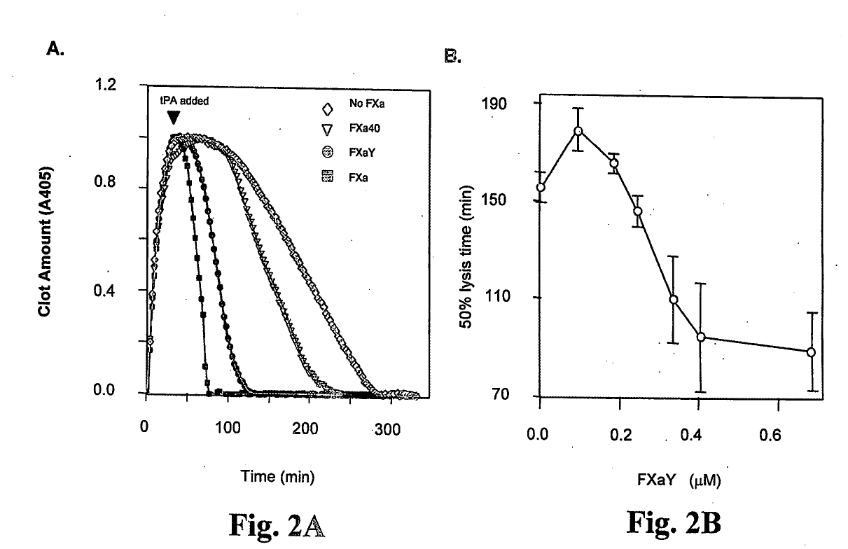

Experiments have been conducted by following lysis of a clot that was formed by adding thrombin (3 nM) to a mixture of fibrinogen (3 μM), Pg (0.6 μM), proPL (100 uM), GEMSA (0.1 μM, a carboxypeptidase B inhibitor) and 2 mM Ca2+, in the presence or absence of Xa, Xaγ or Xa40 (0.6 uM) (see FIG. 2 panel A). Clot formation and subsequent lysis initiated by addition of tPA (10 nM, arrow) were monitored by turbidity. The rate of clot lysis was found to be greatly enhanced in the presence of Xay compared to no Xa composition. In the presence of Xa40, the rate of lysis was only slightly enhanced. Although we have found that Xa40 cannot interact with Pg, the slight enhancing effect can be accounted for by the approximately 10% contamination of Xaγ. When untreated Xa (i.e. a 1:1 mixture of intact Xa (FXaα) and Xa autolytically cleaved to remove a C-terminal 3KDa fragment (Xaβ) is used in this experiment, an even faster rate of lysis is observed than for FXaγ. To determine the concentration range over which Xaγ is functional in this experiment, a titration has been conducted and the time required to achieve 50% lysis was plotted (see panel B). With all other parameters constant, this experiment demonstrates a Xaγ dose dependence on acceleration of clot lysis. As a comparison, the effect is significantly larger than the initial observation leading to the important discovery of thrombin-activated fibrinolysis inhibitor function. It will be appreciated that other concentrations may also be effective in effecting clot lysis depending on the conditions such as pH, temperature and the like as would be obvious to one skilled in the art.

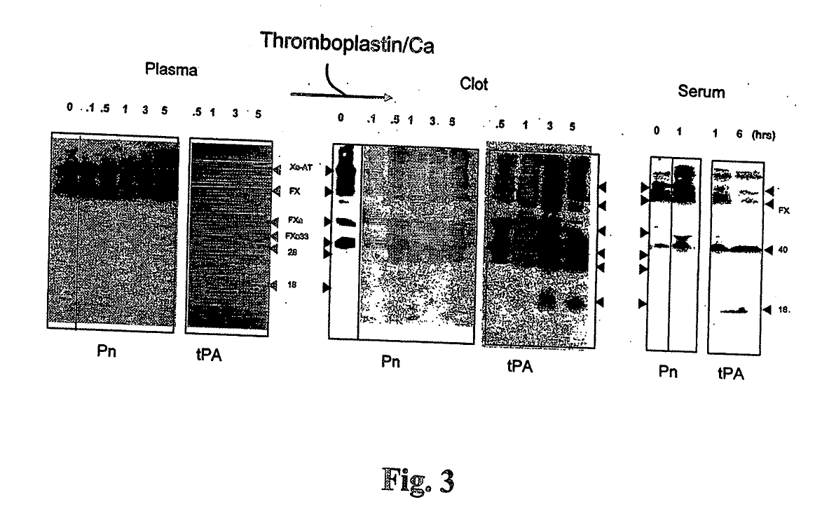

Fragmentation of Factor X and Factor Xa During Clot Lysis in Plasm. To investigate the physiological relevance of Pn-mediated compositions of Xa, experiments were conducted to determine if the fragmentation patterns observed using purified proteins are representative of those formed in the complex plasma milieu. In these experiments, plasma was clotted utilizing thromboplastin as a source of the coagulation initiators, tissue factor and proPL. In this way, Xa is generated in situ. Clot lysis was then initiated by addition of Pn (0.1 μM) or tPA (10 nM). Utilizing a X/Xa heavy chain-specific monoclonal antibody (mAb), we conducted Western blot analyses of plasma, clots and serum. The data (FIG. 3) show that treatment of plasma with Pn (2 μM) or tPA (100 nM) for a period of 5 hours at 25° C. has no effect on the distribution of X-derived bands. The latter is approximately half of the predicted therapeutic dose. Multiples of high molecular weight species observed for each experiment represent covalent Xa-serpin complexes of which the probable Xa-antithrombin (Xa-AT) complex is indicated. The highest band in each gel represents IgG which is weakly detected by the mouse secondary antibody used in the detection system. When clot is then formed by adding thromboplastin (as a source of tissue factor) and calcium (Ca²⁺), it is evident that the majority. of X is converted to Xa, which is at least 50% trapped by AT. Xa33 is visible as a strong band, that in addition to cleavage by Pn, we have observed can be generated much more slowly by autoproteolysis. Since clot formation is allowed to proceed for 30 minutes in these experiments, autoproteolysis could account for part of the production of the Xa33 observed. An important point to note is that Xa33 is recognized by this mAb better than Xa or X and is therefore disproportionately represented. Interestingly, when Pn or tPA is added to the clot, the remaining Xa disappears over time with a concomitant increase in a 28 KDa fragment. Xa33 appears unchanged over the duration of the experiment, but this observation could be only due to the extent of recognition by this mAb. We have previously reported the appearance of this 28 KDa fragment in purified Pn digestions of Xa (Pryzdial, E. L. G. and Kessler, G. E. (1996) Journal of Biological Chemistry 271:16614-16620] as a very minor product. Its. identity has been deduced based on antigenicity, size and known cleavages in X, as Xa33, with an extra cleavage at Arg296. Since this would excise the activation fragment in addition to part of the heavy-chain, the 28 KDa species could be derived from either Xa or X. An additional low molecular weight 18 KDa species was observed, which would be expected to occur in molecules that are not bound to proPL by cleavage at Lys43 in the light-chain. An interesting observation is that the 28 and 18 KDa fragments are produced faster when clot is lysed with tPA than with Pn. These data show that the X/Xa cleavage products observed for purified X/Xa proteins also occur in plasma, and therefore support physiological relevance. To determine which FX/Xa species are released. into serum as the clot is lysed, at various times the dissolving clot was spun and the supernatant was run on gels. In the resulting serum, we observed only FXa-serpin complexes, FX, a 40 KDa and the 18 KDa fragment independent of platelets. The 40 KDa species presumably represents a fragment we have also characterized that is, formed by cleavage at Lys43 when Xa is not bound to proPL. These observations. suggest that nearly all of Xa generated during clot formation in plasma is recruited to the clot. Cumulatively these data support the conclusion that Pn-mediated compositions of Xa and X are generated under physiological conditions. Natural occurrence implies immune tolerance to these peptide bond-modified derivatives of X and Xa, which adds further support for their therapeutic potential.

Thus in one embodiment of the invention there is provided a method for treating patients with conditions necessitating an accelerated dissolution of blood clots. The method involves the administration of a coagulation protein having a basic C-terminal amino acid capable of accelerating the dissolution of blood clots in the presence of intrinsic or therapeutic tissue plasminogen activator.

Conditions that can be treated in accordance with this method are conditions in which a faster rate of clot dissolution is desirable or conditions in which clot dissolution is abnormally low. Such conditions may comprise but are not limited to: thrombosis, platelet hyperactivity, cardiac ischemia, wound, cardiovascular disease, atherosclerosis, myocardial infarction. It will be appreciated that administration of the coagulation protein may be prophylactic to patients susceptible to the above mentioned conditions.

Preferred routes of administration are intravenous, intramuscular, subcutaneous, intraperitoneous, and intraarterial. It will be appreciated that other methods of administration may be used such as, for example, local administration at the site of a clot using a catheter.

The coagulation protein comprising a basic C-terminal amino acid is preferably administered as part of a pharmaceutical composition which may also comprise a pharmaceutically acceptable carrier as would be obvious to one skilled in the art.

It will be appreciated that the coagulation protein of the present invention may be administered concurrently with one or more fibrinolytic agents such as but not limited to tissue plasminogen activator, urokinase, streptokinase and the like.

It will also be appreciated that the coagulation protein of the present invention may be administered concurrently with one or more inhibitor of the coagulation pathway. For example, inhibitors of thrombin, such as but not limited to heparin, bivalirudin, liperudin and the like.

In a further aspect of the invention, detection of derivatives of factor X/Xa, V/Va in patient plasma may serve as a clinical marker for fibrinolytic potential. Detection of the fibrinolytic activity can be achieved by obtaining a blood sample from a patient and measuring the relative concentration or activity of a coagulation protein comprising a basic C-terminal amino acid. It will be appreciated that the coagulation protein may undergo in vivo modification and that accordingly the method also comprises measuring a concentration of coagulation protein derivatives of the coagulation protein comprising a basic C-terminal amino acid.

### INDUSTRIAL APPLICABILITY

The present invention advantageously provides a novel strategy to better initiate clot lysis, while avoiding the complications often associated with current thrombolytic drugs. The products and methods of the present invention provide industrially applicable means for the acceleration of blood clot dissolution, and a method for detecting a fibrinolytic potential in a subject.

The embodiment(s) of the invention described above is(are) intended to be exemplary only. The scope of the invention is therefore intended to be limited solely by the scope of the appended claims.

1. A method for accelerating blood clot dissolution in a subject in need thereof, the method comprising:
a) administering to said subject at least one coagulation protein comprising a basic C-terminal amino acid in an amount effective to dissolve said blood clot.

2. The method as claimed in claim 1 wherein said protein is an anionic phospholipid-binding protein.

3. The method as claimed in claim 1 wherein said subject has a condition selected from: thrombosis, platelet hyperactivity, cardiac ischemia, wound, cardiovascular disease, atherosclerosis, myocardial infarction or a combination thereof.

4. The method as claimed in claim 3 wherein said subject is susceptible to said condition and said administering is prophylactic.

5. The method as claimed in claim 1 wherein said at least one coagulation protein is a derivative of Factor X.

6. The method as claimed in claim 5 wherein said derivative is selected from Factor Xaα, Xaβ, Xaγ, or a combination thereof.

7. The method as claimed in claim 1 wherein said at least one coagulation protein is a derivative of Factor V.

8. The method as claimed in claim 7 wherein said derivative is Factor Va.

9. The method as claimed in claim 1 wherein said at least one coagulation protein comprises a derivative of Factor X and a derivative of factor V.

10. The method as claimed in claim 1 wherein said at least one coagulation protein is a derivative of Factor X and wherein administering comprises administering to the subject a pharmaceutical composition comprising said derivative of Factor X and an acceptable carrier.

11. The method according to claim 10 wherein said derivative of Factor X is selected from Xaα, Xaβ and Xaγ or a combination thereof.

12. The method as claimed in claim 7 wherein administering comprises administering to the subject a pharmaceutical composition comprising said derivative of Factor V and an acceptable carrier.

13. The method according to claim 12 wherein said derivative of Factor V is selected from Va.

14. The method as claimed in claim 1 wherein said pharmaceutical composition further comprises a fibrinolytic agent selected from tissue plasminogen activator, urokinase, streptokinase or a combination thereof.

15. The method as claimed in claim 10 wherein said pharmaceutical composition further comprises an inhibitor of thrombin.

16. The method as claimed in claim 15 wherein said inhibitor of thrombin is selected from hirudin, bivalirudin, lepirudin and heparin or a combination thereof.

17. The method as claimed in claim 14 wherein said pharmaceutical composition is administered intravenously, intramuscularly, subcutaneously, intraperitoneously or intraarterially or a combination thereof.

18. A method for detecting a fibrinolytic potential in a subject the method comprising:
a) obtaining a blood sample from said subject; and
b) measuring a relative concentration of a coagulation protein selected from a coagulation protein comprising a basic C-terminal amino acid, a derivative of a coagulation protein comprising a basic C-terminal amino acid or a combination thereof.

19. The method as claimed in claim 18 wherein said coagulation protein is selected from a derivative of Factor X or Factor V.

20. A pharmaceutical composition comprising a coagulation protein for accelerating blood clot dissolution wherein said coagulation protein comprises a basic C-terminal amino acid.

21. A pharmaceutical composition according to claim 20, wherein said coagulation protein is a derivative of Factor X or Factor V or a combination thereof.

22. A pharmaceutical composition according to claim 21, wherein said Factor X is selected from Xaα, Xaβ and Xaγ or a combination thereof, and Factor V is selected from Va.

23. A pharmaceutical composition according to any one of claims claim 20, and a pharmaceutically acceptable carrier, and/or one or more fibrinolytic agents, and/or one or more inhibitors of the coagulation pathway.

24. The method of claim 10, wherein said at least one coagulation protein is an anionic phospholipid-binding protein.
